(19) United States
(12) Patent Application Publication
(10) Pub. No.: US 2010/0131025 A1
(43) Pub. Date: May 27, 2010

(54) Apparatus and method for treating diabetic neuropathy and peripheral vascular pathologies

(76) Inventor: Shelly Henry, Yukon, OK (US)

Correspondence Address: JAMES M. LOEFFLER, ESQ. PLLC, COURTHOUSE CORPORATE CENTER, 320 CARLETON AVENUE, SUITE 6800, CENTRAL ISLIP, NY, 11722, US

(21) Appl. No.: 12/592,444

(22) Filed: Nov. 24, 2009

Related U.S. Application Data
(60) Provisional application No. 61/118,069, filed on Nov. 26, 2008.

(51) Int. Cl. A61N 1/36; A61N 5/067

(52) U.S. Cl. 607/3

(57) ABSTRACT

The present invention is directed to an apparatus and method for treating a person afflicted with diabetic neuropathy, diabetically-associated wound, peripheral arterial disease (PAD), pain disorders, TMJ, shoulder, migraines, back pain, neck pain but not limited to the disorders mentioned. Can be any neurological disorder, circulatory disorder, lymphatic disorder, any disorder that can be helped by exercise and movement of muscle tissue and stimulation of nerves, improved lymphatic drainage, decreased edema, increased circulation. The apparatus comprises one or more cuffs constructed from straps or bands or embedded pads with cold laser diodes which can be secured about a limb or body part of a patient (e.g., leg and/or arm) and which are constructed with low intensity (cold laser) diodes for applying laser light and with electrical electrodes for applying electrical pulses, respectively, to the patient's limbs, torso, head, neck, arms, hands, feet at the locations of the cuffs or the embedded pads.

### CROSS-REFERENCE TO RELATED APPLICATIONS

This application claims the benefit of U.S. Provisional Patent Application No. 61/118,069 filed Nov. 26, 2008.

### BACKGROUND

Treatments for diseases such as diabetic neuropathy and peripheral arterial disease and related conditions such as diabetic wounds are difficult to treat and few conventional therapies provide significant relief or improvement.

Diabetic neuropathy is manifested in a variety of ways including, for example, loss of coordination and balance, extreme sensitivity to touch, numbness or insensitivity to pain or temperature, tingling, burning, or prickling, sharp pains or cramps, atrophy of muscles, wounds that do not heal well and become easily infected, erectile or sexual dysfunction, loss of bladder or bowel control, abnormal sweating with inability to tolerate changes in temperature, abnormal blood pressure control, causing lightheadedness when standing up, trouble digesting food, double vision, aching behind the eye, inability to focus the eye, pain in the front of the thigh, paralysis on one side of the face (Bell's palsy), problems hearing, severe pain in pelvis or lower back, and chest or abdominal pain sometimes mistaken for angina, heart attack, or appendicitis.

Peripheral arterial disease (PAD) is a circulatory problem in which arteries have become sclerotic, thereby reducing blood flow to the limbs. Peripheral arterial disease causes the extremities, e.g., the legs, to have inadequate blood flow, which causes symptoms, most notably leg pain when walking, a condition called intermittent claudication. Peripheral arterial disease is likely to be a sign of widespread accumulation of fatty deposits in the arteries (atherosclerosis), which may be reducing blood flow to the heart and brain as well. Strictly speaking, peripheral arterial disease refers to a problem with any of the arteries outside, or peripheral to, the heart, but the term is commonly used to describe circulatory problems in the limbs or pelvis. Intermittent claudication is characterized by muscle pain or cramping in the legs or arms that is triggered by a certain amount of activity, such as walking, but disappears after a few minutes of rest. The location of the pain depends on the location of the clogged or narrowed artery. Calf pain is most common. The severity of intermittent claudication varies widely. Pain from this condition can range from mildly bothersome to debilitating. Severe intermittent claudication can impair one's ability to function and engage in any physical activity. Other signs and symptoms of peripheral arterial disease include, leg numbness or weakness, cold legs or feet, sores on the toes, feet or legs that won't heal, a change in the color of the legs, hair loss on the feet and legs, and changes in the nails. If peripheral arterial disease progresses, pain may even occur at rest or when lying down. This is called ischemic rest pain. It may be intense enough to prevent sleep or wake from sleep. One may be able to temporarily relieve the pain by hanging the legs over the edge of the bed or by walking around the room. The most common cause of peripheral arterial disease is atherosclerosis. In atherosclerosis, fatty deposits (plaques) build up in the artery walls and reduce blood flow. As noted, peripheral arterial disease can lead to open sores that don't heal, injury, or infection of the feet and legs, especially in persons who also have diabetes. Critical limb ischemia (CLI) is the extreme case of this condition and can cause tissue death (gangrene), sometimes requiring amputation of the affected limb. Stroke and heart attack are among the more serious and most common complications that accompany peripheral arterial disease. Atherosclerosis causing symptoms of peripheral arterial disease is not limited to the legs. Fat deposits also build up in arteries supplying the heart and brain.

Improved methods for treating these disease conditions would be highly desired.

### BRIEF DESCRIPTION OF THE DRAWINGS

FIG. 1 is a bottom plan view of a therapeutic cuff in accordance with an embodiment of the present invention.

FIG. 2 is a top plan view of a therapeutic cuff of FIG. 1.

FIG. 3 is a side cross-sectional view of the therapeutic cuff of FIG. 1 taken through line 3-3.

FIG. 4 is a front view of legs and a lower torso of a subject wearing several therapeutic cuffs as described herein.

FIG. 5 is a back view of the subject of FIG. 4.

FIG. 6 is a schematic view of an operating control device for the therapeutic cuff of the present invention.

FIG. 7 is a side view of the control device of FIG. 6.

FIG. 8 is a diagram of a subject's head having a therapeutic cuff of the present invention secured thereabout.

### DETAILED DESCRIPTION OF THE INVENTION

The present invention is directed to an apparatus and method for treating a person afflicted with diabetic neuropathy, diabetically-associated wound or peripheral arterial disease (PAD). The apparatus comprises one or more cuffs constructed from straps or bands which can be secured about a limb or body part of a patient (e.g., leg and/or arm) and which are constructed with low intensity (cold laser) diodes for applying laser light and with electrical electrodes for applying electrical pulses, respectively, to the patient's limbs at the locations of the cuffs. This could be used as a pad with the diodes embedded in the pad to hold it in place which would not require the cuff.

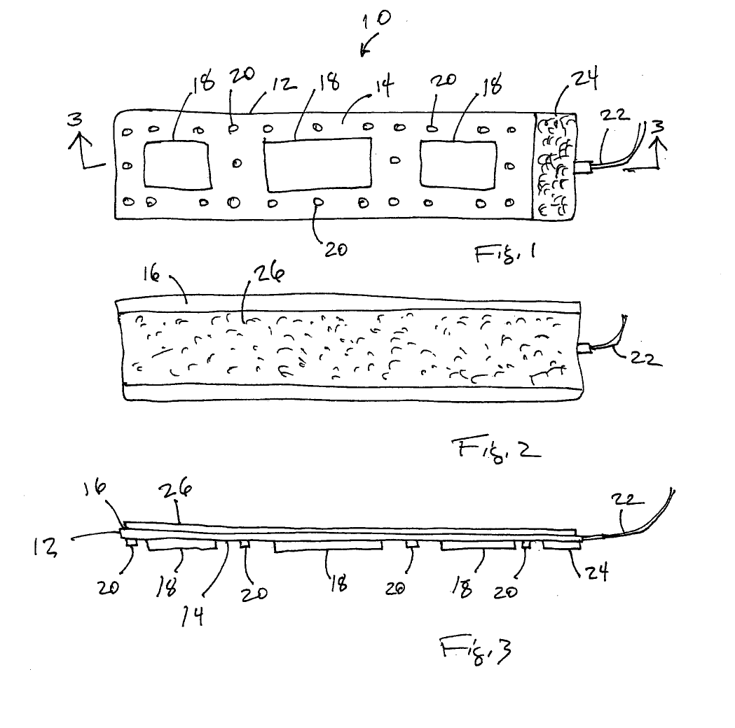

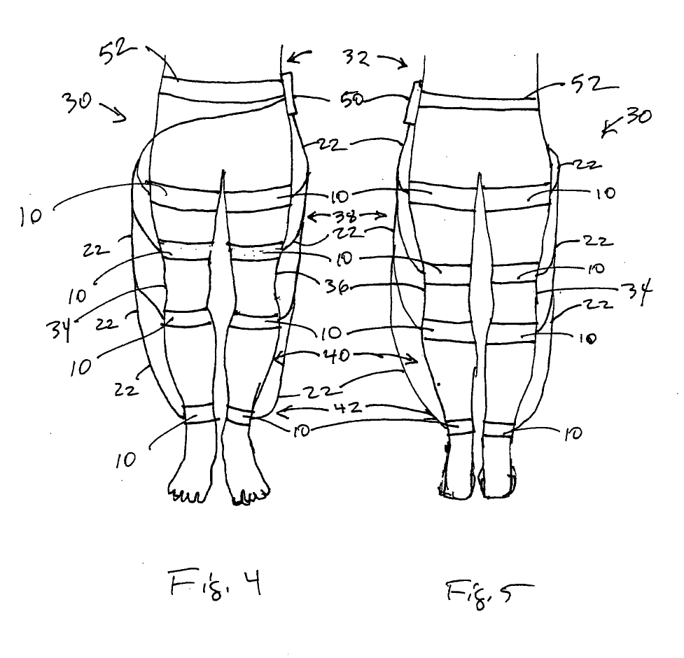

Each therapeutic cuff of the apparatus comprises at least one laser diode and at least one electrode. Preferably there are a plurality of laser diodes and at least two electrode pads disposed on the cuff. One embodiment of the therapeutic cuff is shown in FIG. 1, and designated as therapeutic cuff 10. The therapeutic cuff 10 is shown constructed of a flexible band of material 12 having an inner surface 14 and outer surface 16. Externally positioned on the inner surface 14 are one or more electrodes 18 (FIG. 1 shows three electrodes 18) and a plurality of laser diodes 20 which are positioned around and between the electrodes 18. Each electrode 18 is connected to an embedded internal electrical lead (not shown) which supplies an electrical current and each laser diode 20 is connected to an embedded internal fiber optic lead (not shown) which supplies coherent laser light to each laser diode 20. The electrical leads and fiber optic leads are preferably bundled together in a cable 22 which extends from a terminal portion or intermediate portion of the band of material 12. The therapeutic cuff 10 preferably further comprises a fastening system such as pins, clips, snaps, or hook and loop fasteners such as are known in the art as Velcro® fasteners. For example, the inner surface 14 may have a hook and loop fastener 24 at a terminal portion thereof, and a complementary hook and loop fastener 26 on the outer surface 16 of the band of material 12 for connecting the therapeutic cuff 10 about the limb or body part.

In a preferred embodiment, the electrode 18 is a portion of a Transcutaneous Electrical Nerve Stimulation unit (or TENS unit) which is an electrical device which produces mild electrical signals to stimulate nerves through unbroken skin. The TENS unit comprises a plurality of electrodes, a pulse generator, a small transformer and frequency and intensity controls. Examples of TENS devices and power and intensity levels which may be used herein are shown in U.S. Pat. Nos. 3,817,254 and 6,751,506, which are hereby expressly incorporated by reference herein in their entirety. TENS units are commercially available.

In a preferred embodiment, each laser diode 20 is a low intensity laser diode. The preferred laser light wavelength range is 400-1400 nm, and more preferably 650-950 nm and the irradiation rate is generally 1-1500 mW/cm² and more preferably 10-100 mW/cm². The laser irradiation may be pulsed or continuous and may comprise only one, or both, of the transverse magnetic (TM) polarization mode or the transverse electric (TE) polarization mode of the optical signal. Other optical and power parameters which may be employed during use of the laser diode 20 of the present invention may be found in U.S. Pat. Nos. 5,445,146 and 6,494,900 which are hereby expressly incorporated herein by reference in their entireties.

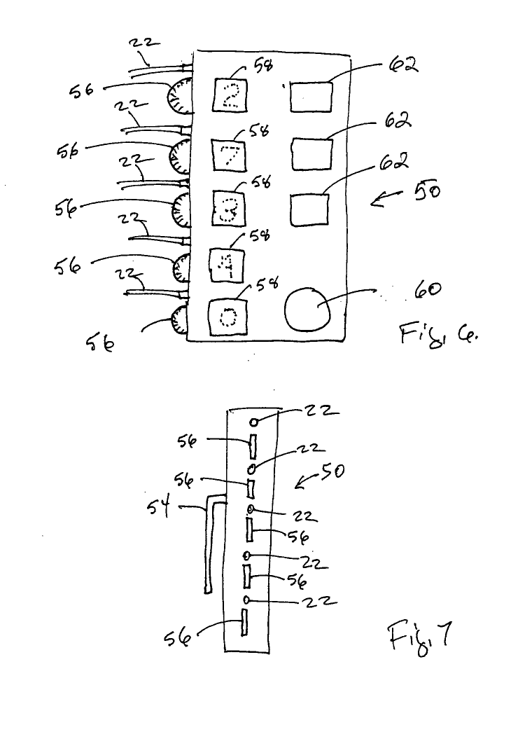

Shown in FIGS. 4 and 5 is a subject 30 having a lower torso 32, a right leg 34 and a left leg 36. A plurality of the therapeutic cuffs 10 are positioned on each of the right leg 34 and left leg 36 of the subject 30 in positions on the thigh area 38, the cuff area 40, and the upper ankle area 42. Preferably the therapeutic cuffs 10 are placed over arteries, veins and lymph nodes or vessels in the right leg 34 and left leg 36. In an alternative embodiment, therapeutic cuffs 10 are placed only on the right leg 34 or the left leg 36 (not both) or may be suitably placed on an arm or the arms of a subject (not shown) along with those on the legs or in substitution thereof.

The positions of the therapeutic cuffs 10 in FIGS. 4 and 5 are considered to be merely exemplary of a variety of specific positions which may be used. Preferably the therapeutic cuffs 10 are positioned so as to place the electrodes 18 and laser diodes 20 over or adjacent arteries, veins, and lymph vessels in the limbs. Further, the electrodes 18 may be positioned over muscles in the limbs.

The therapeutic cuff 10 of the present invention in an alternate embodiment could be secured about (1) the head, for treating migraine headaches, TMJ, stress headaches (e.g., FIG. 8), (2) the back of the neck, for treating neck pain, (3) the back, for treating back pain, (4) shoulder for treating shoulder pain and tightness, (5) Chest for treating chest pain and tightness of muscles, (6) Wrist for treating pain and tightness, (7) Hand for treating pain and tightness, (7) Hip for treating hip pain and tightness, (8) Knee for treating knee pain and tightness. Units will be designed to accommodate the specific body part that will be treated for the desired effect.

Without wishing to be held to theory, it is thought that the low intensity laser irradiation from the laser diodes 20 has its effect in strengthening arterial and venous vessel walls and constriction thereof thereby reducing swelling in the limbs (edema) thereby resulting in an acute improvement in blood flow and a reduction in pain and swelling. When laser is used alone, these effects tend to be temporary, i.e., the improvements generally last only a few days or weeks. Further, without wishing to be bound by theory, electrical stimulation alone appears to cause stimulation of muscles in and surrounding blood vessels thereby helping to increase blood flow therethrough.

When both laser light and electrical stimulation is used in the present invention, the laser and electrical stimulation act synergistically to cause significantly longer lasting improvements, even to the point that subjects having severe cases of diabetic neuropathy and PAD who could not walk prior to the treatment contemplated herein are able to walk and even exercise after treatment. For example, when laser or electrical stimulation is used alone there may be an increase of 10 mm of basal blood pressure, whereas when laser and electrical stimulation are used simultaneously together (as contemplated herein) they act synergistically, for example, the resulting increase in blood pressure can be from 25-75 mm.

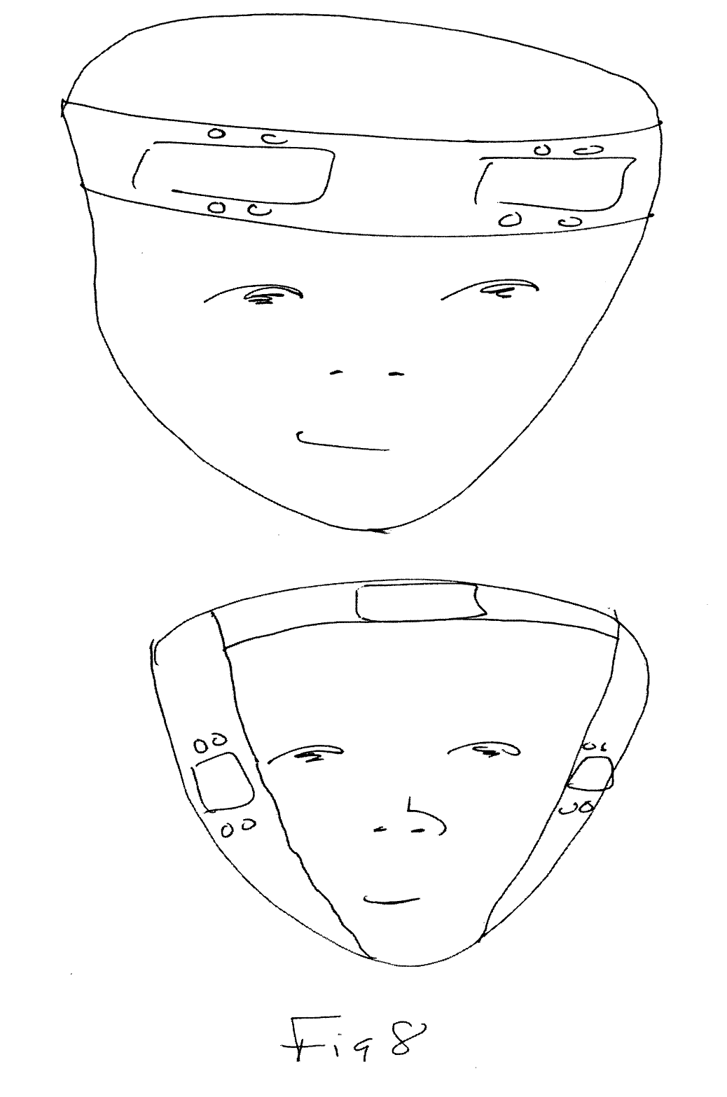

In a preferred embodiment, as shown in FIGS. 4-7, the cables 22 which lead from each therapeutic cuff 10 are operatively connected to an operating control and power device 50 which is clipped to a belt 52 or other device by a clip 54 (or other holding device). The control and power device 50 has a power source such as a battery or AC current and which has an optical signal generator for providing the laser signals to each therapeutic cuff 10. Power controls are represented on control device 50 by dials 56 which can be turned to regulate the power and/or intensity of the optical and/or electrical signals wherein the power or intensity level is shown in LED windows 58. An on/off button switch 50 is represented as well as signal buttons 62 which can be turned on or off to cause the optical and/or electrical signals to be emitted in specific configurations such as “pulsing”, “continuous”, “massaging”, “tapping”, or other desired modes.

Treatment protocols vary depending on the particular affliction experienced by the subject and the magnitude or severity thereof. For example, a single treatment may last for 5 to 30 minutes, and may be repeated one to several times/day, or week. For example, a subject may be given a single treatment or several each day for two to four weeks or until relief is attained. The intensity of the laser and electrical signals may be those described elsewhere herein. The entire content of each patent or published application cited herein is expressly incorporated herein by reference in its entirety.

The present invention is not to be limited in scope by the specific embodiments described herein, since such embodiments are intended as but single illustrations of one aspect of the invention and any functionally equivalent embodiments are within the scope of this invention. Indeed, various modifications of the methods of the invention in addition to those shown and described herein will become apparent to those skilled in the art form the foregoing description.

What is claimed is:

1. An apparatus for treating neuropathy and vascular pathologies comprising:
at least one cuff member configured to be secured about a body part of a patient;
the at least one cuff member being constructed with at least one low intensity diode for applying laser light and with at least one electrical electrode for applying electrical pulses, respectively, to the body part of the patient adjacent to the at least one cuff member.

2. The apparatus for treating neuropathy and vascular pathologies as recited in claim 1 wherein the at least one cuff is constructed from a strap.

3. The apparatus for treating neuropathy and vascular pathologies as recited in claim 1 wherein the at least one low intensity diode is a cold laser.

4. The apparatus for treating neuropathy and vascular pathologies as recited in claim 1 wherein the at least one cuff member is constructed with a plurality of low intensity diodes for applying laser light and a plurality of electrical electrodes for applying electrical pulses.

5. The apparatus for treating neuropathy and vascular pathologies as recited in claim 1 further comprising an electrical lead connected to the at least one electrical electrode for supplying the at least one electrical electrode with an electrical current.

6. The apparatus for treating neuropathy and vascular pathologies as recited in claim 1 further comprising a fiber optic lead connected to the at least one low intensity diode for supplying the at least one low intensity diode with a laser light.

7. The apparatus for treating neuropathy and vascular pathologies as recited in claim 1 wherein the at least one cuff member further comprises a fastening system such as at least one of pins, clips, snaps, and hook and loop fasteners.

8. The apparatus for treating neuropathy and vascular pathologies as recited in claim 1 wherein the at least one electrical electrode is a portion of a Transcutaneous Electrical Nerve Stimulation unit.

9. The apparatus for treating neuropathy and vascular pathologies as recited in claim 8 wherein the Transcutaneous Electrical Nerve Stimulation unit comprises a plurality of electrodes, a pulse generator, a small transformer and frequency and intensity controls.

10. The apparatus for treating neuropathy and vascular pathologies as recited in claim 1 wherein the laser light is applied in the laser light wavelength range of about 400 nm to about 1400 nm.

11. The apparatus for treating neuropathy and vascular pathologies as recited in claim 1 wherein the electrical pulses are applied to achieve an irradiation rate in the range of about 1 mW/cm² to about 1500 mW/cm².

12. The apparatus for treating neuropathy and vascular pathologies as recited in claim 1 wherein the laser irradiation is continuous.

13. The apparatus for treating neuropathy and vascular pathologies as recited in claim 1 wherein the laser irradiation comprises at least one of the transverse magnetic polarization mode and the transverse electric polarization mode of the optical signal.

14. A method of treating neuropathy and vascular pathologies comprising the steps of:
securing at least one cuff member having at least one low intensity diode and at least one electrical electrode adjacent to a body part of a patient;
applying laser light and applying electrical pulses via the at least one low intensity diode and at least one electrical electrode, respectively, to the body part of the patient adjacent to the at least one cuff member.

15. The method for treating neuropathy and vascular pathologies as recited in claim 14 wherein the laser light is applied in the laser light wavelength range of about 400 nm to about 1400 nm.

16. The method for treating neuropathy and vascular pathologies as recited in claim 14 wherein the electrical pulses are applied to achieve an irradiation rate in the range of about 1 mW/cm² to about 1500 mW/cm².

17. The method for treating neuropathy and vascular pathologies as recited in claim 14 further comprising the step of varying a treatment protocol according to specific needs of the patient.

18. The method for treating neuropathy and vascular pathologies as recited in claim 14 wherein the laser irradiation comprises at least one of the transverse magnetic polarization mode and the transverse electric polarization mode of the optical signal.
